(19) United States
(12) Patent Application Publication
(10) Pub. No.: US 2015/0065509 A1
(43) Pub. Date: Mar. 5, 2015

(54) HIGHLY 6-SUBSTITUTED -2,4-DIAMINOPYRIMIDINES AS INHIBITORS OF ANTHRAX

(71) Applicants: RAMNARAYAN KALYANARAMAN, (US); THE BOARD OF REGENTS FOR OKLAHOMA STATE UNIVERSITY, STILLWATER, OK (US)

(72) Inventors: William Barrow, Stillwater, OK (US); Christina Renee Bourne, Stillwater, OK (US); Richard Alan Bunce, Stillwater, OK (US); Kenneth D. Berlin, Stillwater, OK (US); Baskar Nammalwar, Stillwater, OK (US); Kalyanaraman Ramnarayan, San Diego, CA (US)

(21) Appl. No.: 14/385,599

(22) PCT Filed: Mar. 15, 2013
(86) PCT No.: PCT/US13/32628

§ 371 Date: Sep. 16, 2014

Related U.S. Application Data
(60) Provisional application No. 61/611,884, filed on Mar. 16, 2012.

(51) Int. Cl. C07D 403/10; C07D 239/49; A01N 43/58

(52) U.S. Cl. CPC C07D 403/10; A01N 43/58; C07D 239/49
USPC 514/248; 544/237; 544/325; 435/184

(57) ABSTRACT

2,4-diaminopyrimidine compounds of generic Formula 1, where R and R′ may be the same or different and are independently selected from: C₁-C₆ alkyl or alkenyl groups with 1, 2, 3, 4, 5 or 6 carbon atoms, which may be: branched or unbranched; saturated or unsaturated; and may or may not be substituted, are used to treat anthrax.

### CROSS-REFERENCE TO RELATED APPLICATIONS

This application claims benefit of U.S. provisional application 61/611,884, filed Mar. 16, 2012, the complete contents of which is hereby incorporated by reference.

### STATEMENT REGARDING FEDERALLY SPONSORED RESEARCH OR DEVELOPMENT

The Government of the United States of America has certain rights in this invention pursuant to 1R01AI-090685 awarded by the National Institutes of Health through the National Institute Allergy and Infectious Diseases.

### BACKGROUND OF THE INVENTION

1. Field of the Invention

The present invention relates to the treatment of anthrax infections. In particular, the invention provides 2,4-diaminopyrimidine compounds for the manufacture of medicaments for use in the treatment of anthrax.

2. Background

Anthrax is a highly infectious disease that normally affects animals, for example goats, cattle, sheep or horses, but which can be transmitted to humans by contact with infected animals, infected animal products or Bacillus anthracis spores.

The transmitter of anthrax is a bacterium called Bacillus anthracis, an encapsulated Gram-positive, nonmotile, aerobic, spore-forming bacterium. Its spores resist destruction and remain viable in the soil and in animal products for years, even for decades.

Humans are usually infected through the skin or from eating meat contaminated with anthrax resulting in cutaneous or gastrointestinal forms of anthrax infections. Substantial danger may also come from the spores of anthrax, which, once inhaled, can result in a disease in the lungs referred to as pulmonary anthrax or also as woolsorter's disease and which is usually fatal.

While anthrax is rare in humans in developed industrialized countries, it still occurs in less developed countries. Furthermore, there is great concern about anthrax as a potential agent of biological warfare and bioterrorism.

Today, antibiotics are given to unvaccinated individuals exposed to anthrax via inhalation. Penicillin, tetracyclines and fluoroquinolones are known to be effective if administered within about 24 hours. Ciprofloxin is approved by the FDA for a postexposure treatment of inhalational anthrax.

Nevertheless, there is great interest and an ongoing need to develop new antibacterial drugs for treating anthrax, for example, as an alternative for fighting strains of Bacillus anthracis which are or which become resistant to the antibiotics that are presently available.

### BRIEF DESCRIPTION OF THE DRAWINGS

FIG. 1 is an illustration of a compound of the present disclosure.

FIG. 2 illustrates one embodiment of a reaction scheme for producing various compounds according to the present disclosure.

FIG. 3 illustrates another embodiment of a reaction scheme for producing various compounds according to the present disclosure.

FIG. 4 is an illustration of a compound derived according to the present disclosure.

FIG. 5 is an illustration of another compound derived according to the present disclosure.

FIG. 6 is an illustration of another compound derived according to the present disclosure.

FIG. 7 is an illustration of another compound derived according to the present disclosure.

FIG. 8 is an illustration of another compound derived according to the present disclosure.

FIG. 9 is an illustration of another compound derived according to the present disclosure.

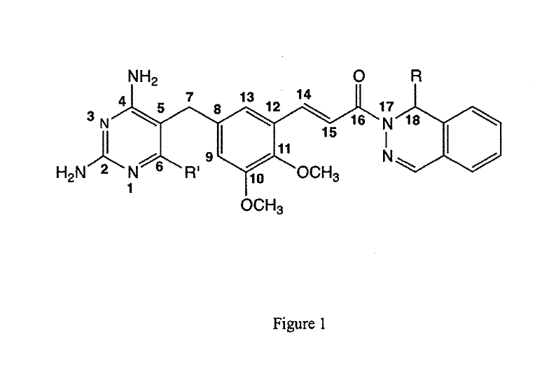

### SUMMARY OF THE INVENTION

Compounds of generic Formula 1:

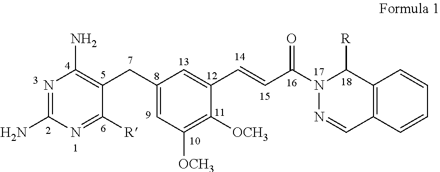

are provide for use in killing the anthrax bacillus B. anthracis and preventing and/or treating infections cause by B. anthracis. Methods of manufacturing the compounds are also provided.

The invention provides compounds of Formula 1:

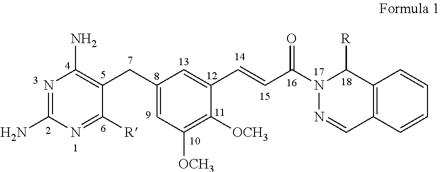

wherein: R and R′ may be the same or different and are independently selected from: C₁-C₆ alkyl or alkenyl groups with 1, 2, 3, 4, 5 or 6 carbon atoms, which may be: branched or unbranched; saturated or unsaturated; and may or may not be substituted. Isomers, pharmacologically acceptable salts, solvates, and hydrates of the compounds are also encompassed. In some aspects, R and R′ are selected from methyl, ethyl, propyl, isopropyl, n-butyl, isobutyl, sec-butyl, tert-butyl, n-pentyl, isopentyl, neopentyl, hexyl, 2-methylpentyl, 3-methylpentyl, 2,3-dimethylbutyl; 2,2-dimethylbutyl, vinyl groups and allyl groups. In some aspects, R is n-propyl or isobutenyl; and in other aspects, R′ is methyl, ethyl or n-propyl. The compound may be, for example:

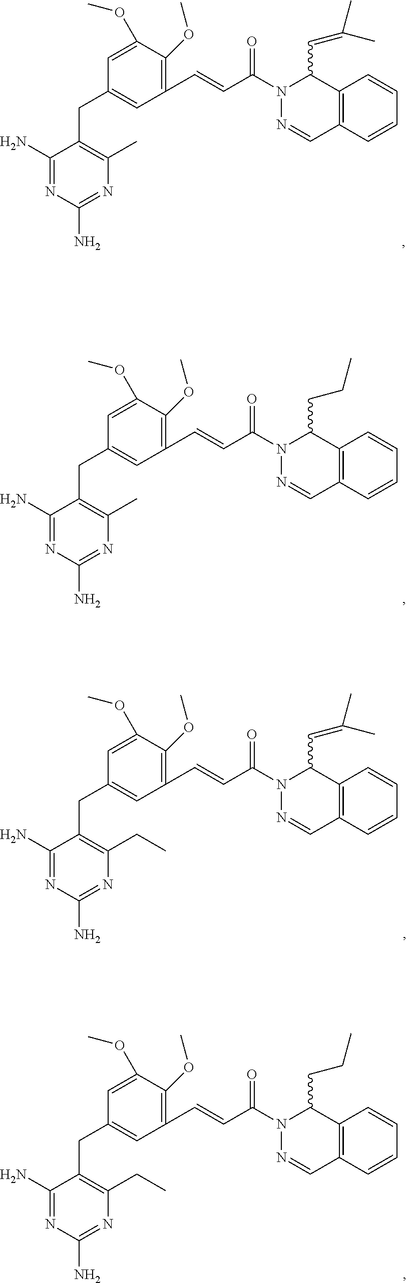 [SMILES: COc1cc(Cc2c(C)nc(N)nc2N)cc(/C=C/C(=O)N2N=Cc3ccccc3C2C=C(C)C)c1OC ; CCCC1c2ccccc2C=NN1C(=O)/C=C/c1cc(Cc2c(C)nc(N)nc2N)cc(OC)c1OC ; CCc1nc(N)nc(N)c1Cc1cc(/C=C/C(=O)N2N=Cc3ccccc3C2C=C(C)C)c(OC)c(OC)c1 ; CCCC1c2ccccc2C=NN1C(=O)/C=C/c1cc(Cc2c(N)nc(N)nc2CC)cc(OC)c1OC]

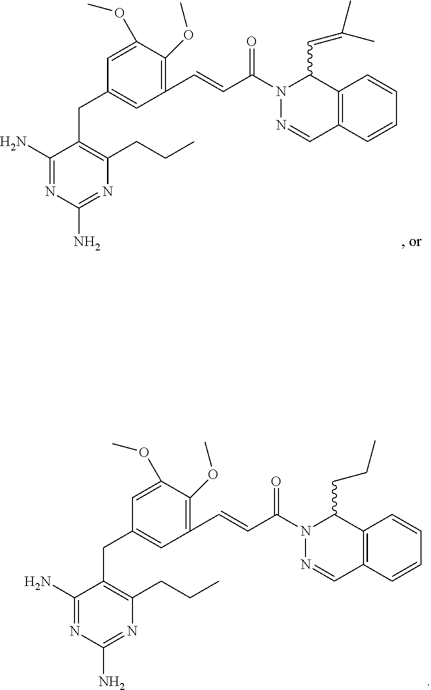 [SMILES: CCCc1nc(N)nc(N)c1Cc1cc(/C=C/C(=O)N2N=Cc3ccccc3C2C=C(C)C)c(OC)c(OC)c1 ; CCCc1nc(N)nc(N)c1Cc1cc(/C=C/C(=O)N2N=Cc3ccccc3C2CCC)c(OC)c(OC)c1]

Also provided are methods of preventing or treating an anthrax infection in a subject in need thereof. The methods comprise a step of administering to the subject a therapeutically effective amount of a compound of Formula 1:

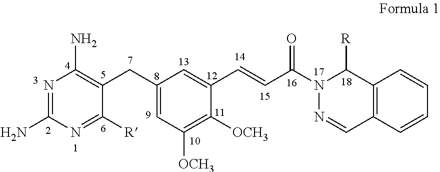

wherein: R and R′ may be the same or different and are independently selected from: C₁-C₆ alkyl or alkenyl groups with 1, 2, 3, 4, 5 or 6 carbon atoms, which may be: branched or unbranched; saturated or unsaturated; and may or may not be substituted; or an isomer, pharmacologically acceptable salt, solvate, or hydrate thereof; and a pharmaceutically compatible carrier may be used. In some aspects, R and R′ are selected from methyl, ethyl, propyl, isopropyl, n-butyl, isobutyl, sec-butyl, tert-butyl, n-pentyl, isopentyl, neopentyl, hexyl, 2-methylpentyl, 3-methylpentyl, 2,3-dimethylbutyl; 2,2-dimethylbutyl, vinyl groups and allyl groups. R may be n-propyl or isobutenyl. R′ may be methyl, ethyl or n-propyl. The compound that is administered may be, for example, one or more of:

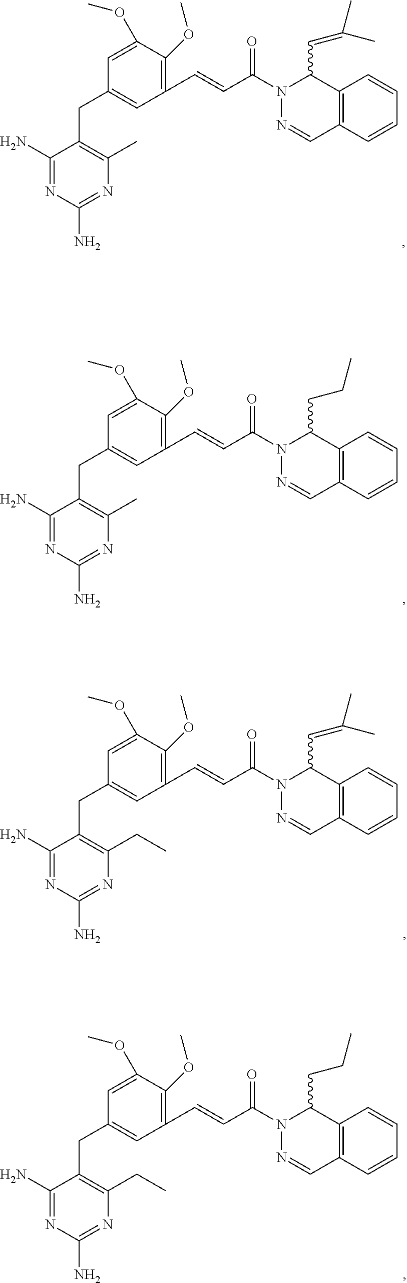 [SMILES: COc1cc(Cc2c(C)nc(N)nc2N)cc(/C=C/C(=O)N2N=Cc3ccccc3C2C=C(C)C)c1OC ; CCCC1c2ccccc2C=NN1C(=O)/C=C/c1cc(Cc2c(C)nc(N)nc2N)cc(OC)c1OC ; CCc1nc(N)nc(N)c1Cc1cc(/C=C/C(=O)N2N=Cc3ccccc3C2C=C(C)C)c(OC)c(OC)c1 ; CCCC1c2ccccc2C=NN1C(=O)/C=C/c1cc(Cc2c(N)nc(N)nc2CC)cc(OC)c1OC]

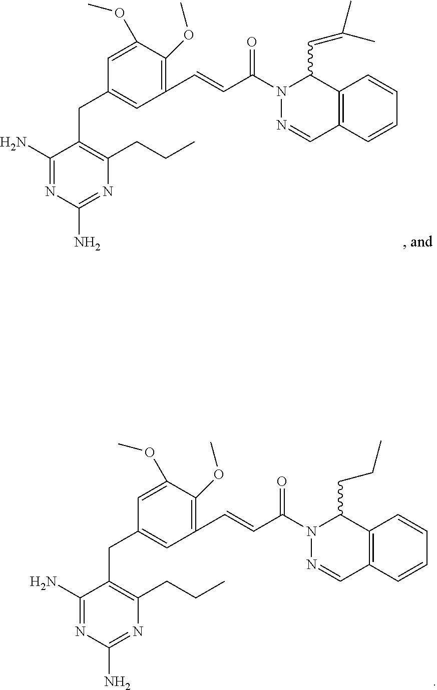 [SMILES: CCCc1nc(N)nc(N)c1Cc1cc(/C=C/C(=O)N2N=Cc3ccccc3C2C=C(C)C)c(OC)c(OC)c1 ; CCCc1nc(N)nc(N)c1Cc1cc(/C=C/C(=O)N2N=Cc3ccccc3C2CCC)c(OC)c(OC)c1]

Further provided are methods of killing Bacillus anthracis. The methods comprise

contacting the Bacillus anthracis with a lethal amount of a compound of Formula 1:

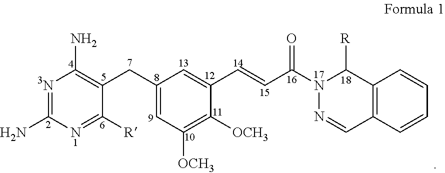

In Formula 1, R and R′ may be the same or different and are independently selected from: C₁-C₆ alkyl or alkenyl groups with 1, 2, 3, 4, 5 or 6 carbon atoms, which may be: branched or unbranched; saturated or unsaturated; and may or may not be substituted; and isomers, pharmacologically acceptable salts, solvates, and/or hydrates of the compound of Formula 1 may be employed.

Also provided are methods of inhibiting dihydrofolate reductase (DHFR). The methods comprise a step of contacting said DHFR with an amount of at least one compound of Formula 1:

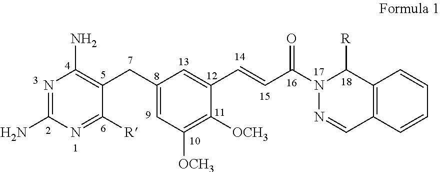

wherein R and R′ may be the same or different and are independently selected from: C₁-C₆ alkyl or alkenyl groups with 1, 2, 3, 4, 5 or 6 carbon atoms, which may be: branched or unbranched; saturated or unsaturated; and may or may not be substituted; or contacting the DHFR with an isomer, pharmacologically acceptable salt, solvate, or hydrate of a compound of Formula 1. The amount of the compound of Formula 1 that is used is sufficient to inhibit the DHFR.

Further provided are methods of synthesizing a compound of Formula 1,

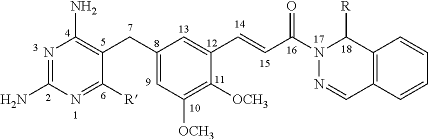

wherein R and R′ may be the same or different and are independently selected from: C₁-C₆ alkyl or alkenyl groups with 1, 2, 3, 4, 5 or 6 carbon atoms, which may be: branched or unbranched; saturated or unsaturated; and may or may not be substituted; as well as isomer, pharmacologically acceptable salt, solvate, or hydrate thereof. The method comprises combining, in a suitable solvent,
i) a compound of Formula 2

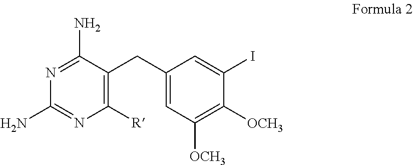

wherein

R′ is selected from: C₁-C₆ alkyl or alkenyl groups with 1, 2, 3, 4, 5 or 6 carbon atoms, which may be: branched or unbranched; saturated or unsaturated; and may or may not be substituted; and

ii) a compound of Formula 3,

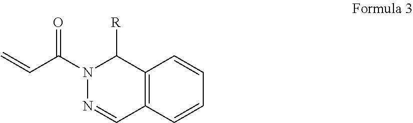

wherein
R is selected from: C₁-C₆ alkyl or alkenyl groups with 1, 2, 3, 4, 5 or 6 carbon atoms,

which may be: branched or unbranched; saturated or unsaturated; and may or may not be substituted. The step of combining is carried out under conditions that permit a reaction to occur between the compound of Formula 2 and the compound of Formula 3 to generate the compound of Formula 1. Such conditions include carrying out the reaction in the presence of a catalyst and at a temperature of 140° C. In some aspects, the catalyst is a Pd catalyst. In some aspects, the suitable solvent is dimethylformamide, 1-ethylpiperidine of a combination of dimethylformamide and 1-ethylpiperidine. R and R′ may be methyl, ethyl, propyl, isopropyl, n-butyl, isobutyl, sec-butyl, tert-butyl, n-pentyl, isopentyl, neopentyl, hexyl, 2-methylpentyl, 3-methylpentyl, 2,3-dimethylbutyl; 2,2-dimethylbutyl, a vinyl group or an allyl group. For example, R may be n-propyl or isobutenyl; and R′ may be methyl, ethyl or n-propyl.

Further provided are compounds of Formula 2

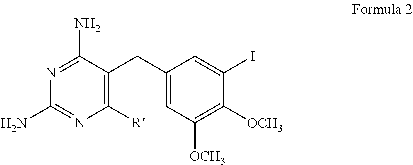

wherein R′ is selected from CH₃, CH₃CH₂ and CH₃CH₂CH₂.

### DETAILED DESCRIPTION OF THE PREFERRED EMBODIMENTS

In one aspect, the invention provides compounds of generic Formula 1:

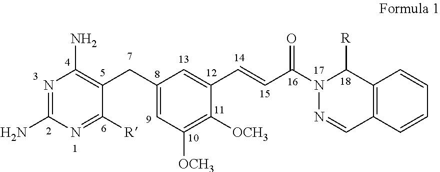

and their use to kill B. anthracis and to treat anthrax infections caused by B. anthracis. The invention also provides methods of manufacturing the compounds.

The 2,4-diaminopyrimidines derivatives (variants) are substituted at carbon-6 of the pyrimidine ring (R′ in Formula 1) and at position 18 (R in Formula 1). R and R′ may be the same or different and are independently selected from: C₁-C₆ alkyl or alkenyl groups with 1, 2, 3, 4, 5 or 6 carbon atoms, which may be: branched or unbranched; saturated or unsaturated (i.e. may have one or more than one C═C double bond); and may or may not be substituted. Exemplary alkyl groups include but are not limited to, e.g. methyl, ethyl, propyl, isopropyl, butyl, isobutyl, sec-butyl, tert-butyl, pentyl, isopentyl, neopentyl, hexyl, 2-methylpentyl, 3-methylpentyl, 2,3-dimethylbutyl; 2,2-dimethylbutyl, etc. Exemplary alkenyl groups include but are not limited to any moiety with at least 2, 3, 4, 5 or 6 carbon atoms in which at least one C═C double bond is present, e.g. various vinyl and allyl groups, CH₂CH₂ (ethylene), etc. In some cases, R is n-propyl or isobutenyl and R′ is methyl, ethyl or n-propyl n-butyl, isobutyl, n-pentyl, 2-methylbutyl, and tertiary butyl. Also included are stereoisomers, enantiomers, and structural isomers of these C₁-C₆ alkyl and/or alkenyl groups. Further, racemates (racemic mixtures) and pure R or S chiral systems of the compounds are encompassed. In addition, various solvates, hydrates, and salt forms of the compounds (e.g. as shown by elemental analysis) are contemplated. All such variants and forms of the compounds of generic Formula 1 are encompassed by the invention, so long as they retain anti-anthrax activity (i.e. anti-Bacillus anthracis activity) as described herein.

R and R′ equivalents may be referred to herein as “groups”, “substituents”, etc., and it is understood that when combined present as part of the molecule represented as Formula 1, at least one atom (usually an H atom) is lost due to the formation of a bond with the atom of the molecule to which R or R′ is attached.

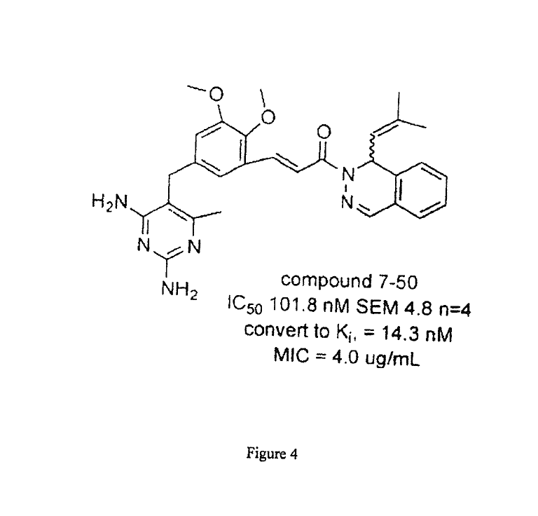

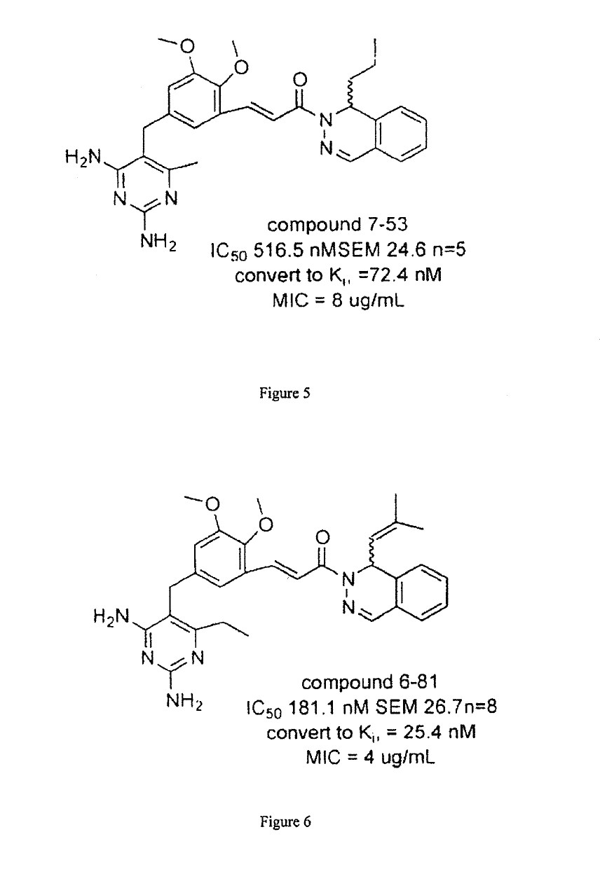

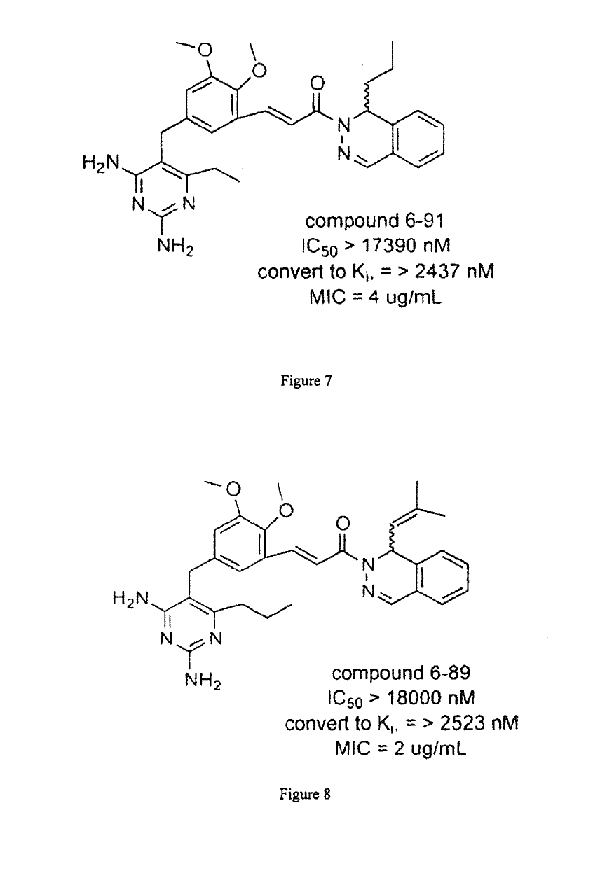

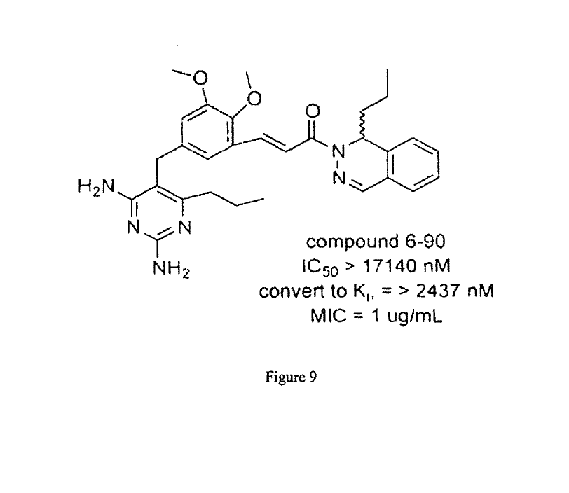

Exemplary compounds are depicted in FIGS. 4-9.

The present invention provides compositions for use in treating anthrax and/or blocking the activity of Bacillus anthracis and/or in killing Bacillus anthracis. The compositions include one or more substantially purified compounds as described herein, and a pharmacologically suitable carrier. The preparation of such compositions for use as medicaments is well known to those of skill in the art. Typically, such compositions are prepared either as liquid solutions, washes, or suspensions, however solid forms such as tablets, pills, powders and the like are also contemplated. Solid forms suitable for solution in, or suspension in, liquids prior to administration may also be prepared. The preparations may also be emulsified. The active ingredients may be mixed with excipients which are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. In addition, the composition may contain other beneficial biologically active ingredients, e.g. anesthetics, other antibiotics, etc. If it is desired to administer an oral form of the composition, various thickeners, flavorings, diluents, emulsifiers, dispersing aids or binders and the like may be added. If cutaneous (topical) application is desired, various creams, sprays, foams or washes may be formulated. For application to the lungs, various inhalable formulations may be prepared e.g. mists, droplets, vapors, etc. The composition of the present invention may also contain any suitable additional ingredients so as to provide the composition in a form suitable for administration. The final amount of compound(s) in the formulations may vary. However, in general, the amount in the formulations will be from about 1-99%, wt/vol or wt/wt, so as to achieve a level in circulation and/or and at the site of infection that is at or above the minimal inhibitory concentration (MIC).

The compositions (preparations) of the invention may be administered by any of the many suitable means which are well known to those of skill in the art, including but not limited to by injection, inhalation, orally, intravaginally, intranasally, topically, as eye drops, via sprays, etc. In some embodiments, the mode of administration is intravenous or by injection (e.g. especially for acute cases), or orally (e.g. for gastrointestinal infections); or topically if the anthrax infection is cutaneous; or via inhalation if the subject's lungs are infected; or by any combination of these. In addition, the compositions may be administered in conjunction with other treatment modalities such as substances that boost the immune system, various chemotherapeutic agents (e.g. raxibacumab), various antibiotic agents (e.g. fluoroquinolones like ciprofloxacin, doxycycline, erythromycin, vancomycin, penicillin, etc.), and the like. Other treatments may also be beneficial, e.g. treatments for particular symptoms that develop such as fever, respiratory difficulty, etc. Treatment may be systemic of targeted to a particular organ or organ system. The compounds may be advantageously used in situations where antibiotics are not effective, e.g. if the bacterium is resistant to antibiotic therapy. Or the compounds may be use as an adjunct treatment with antibiotics, or as the sole administered agent.

The amount of compound that is administered may vary from subject to subject, and may be determined e.g. via clinical trials. The amount and method of administration may depend on the weigh, gender, age, generally physical condition, stage of the illness, etc. of the subject. The precise amount that is administered and the protocol of administration (e.g. the frequency, means, etc.), is generally established by a medical professional such as a physician. Generally, the amount will be in the range of from about 0.2 g to about 2 g of compound per kg of body weight of an adult subject, or about 0.5 to about 2 g of compound per kg of body weight of an adult subject.

The subjects to whom the compounds of the invention are administered are generally mammals and may be humans or non-humans. Infected humans may be those that are inadvertently exposed to and/or which ingest or come into contact with anthrax (usually anthrax spores) through eating or otherwise contacting objects/surfaces contaminated with anthrax spores in the wake of a previous infection (e.g. animal carcasses, waste material, etc.). For non-human, veterinary applications, the subjects may be any of those which are susceptible to infection, including wild and domesticated herbivorous mammals that ingest or inhale the spores. Some examples include various livestock such as cattle, sheep goats, etc. In other instances, the contact with the spores may be purposeful on the part of another, i.e. due to deliberate exposure caused by terrorist activity or as the result of government sanctioned chemical warfare, etc.

The compounds (agents) described herein have been shown to inhibit dihydrofolate reductase, an enzyme critical in anthrax replication. As such, they may be used to kill the causative agent of anthrax, B. anthracis, both in vitro and in vivo (e.g. within a subject). In some aspects, the compounds are used to kill, destroy, or otherwise damage, and/or prevent reproduction of the bacterium (either in vitro or in vivo). In general, the IC₅₀ of a compound that is used is in the range of from about 0.01 to about 20,000, or about 0.5 to about 1000, or about 1.0 to about 500, or about 10 to about 200, 100 or 50 mM. In some embodiments, the IC₅₀ is in the μM range, e.g. from about 5 to about 1000, or about 10 to about 500, or about 10 to about 200, 100 or 50 μM. The agents may also be used to prevent the onset of infection (e.g. to prevent the appearance of symptoms associated with anthrax) and/or to treat known or existing anthrax infections and/or the spread of infection caused by B. anthracis. The compounds may be administered prophylactically, e.g. when infection is possible or is likely to occur, or suspected to have occurred but prior to the onset of overt, detectable symptoms; or after infection is known to be present, (e.g. after the onset of overt, detectable symptoms), regardless of the stage or precise location of the infection. Symptoms of anthrax that may be treated using the compounds of the invention include but are not limited to: skin, mouth or gastrointestinal lesions; fever; cold or flu-like symptoms; difficulty breathing; gastrointestinal distress; vomiting of blood; severe diarrhea; acute inflammation of the intestinal tract; loss of appetite, etc. The compounds described herein may be administered to treat any of these symptoms.

By “treating” anthrax or symptoms thereof, we mean that the presence or degree of symptoms of the disease, including death, is decreased compared to untreated subjects having a similar affliction. Typically, the spread of infection and attendant symptoms may be slowed and is usually eventually reversed due to the treatment. Death may be avoided.

The invention also provides methods of inhibiting the enzyme dihydrofolate reductase (DHFR). The method involves exposing the DHFR to one or more of the compounds of the invention, in an amount to inhibit an activity DHFR. Upon contact and binding to the compound, the enzymatic activity of the DHFR is generally slowed or decreased by at least by about 25, 30, 35, 40, 45, 50, 55, 60, 65, 70, 75, 80, 85, 90 or even 100% (i.e. completely inhibited) of the level of DHFR that is not contacted by the compound. Inhibition of DHFR may be in vitro or in vivo.

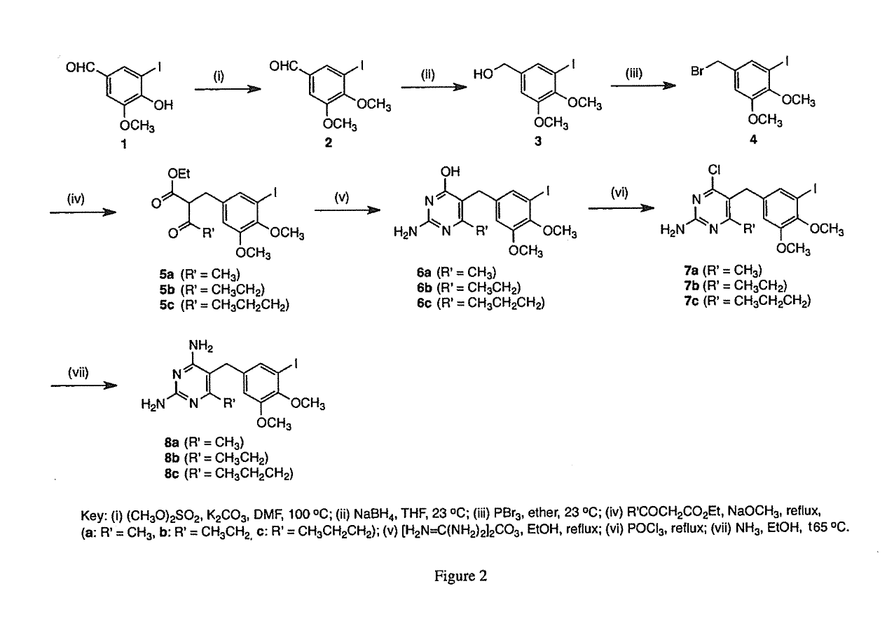

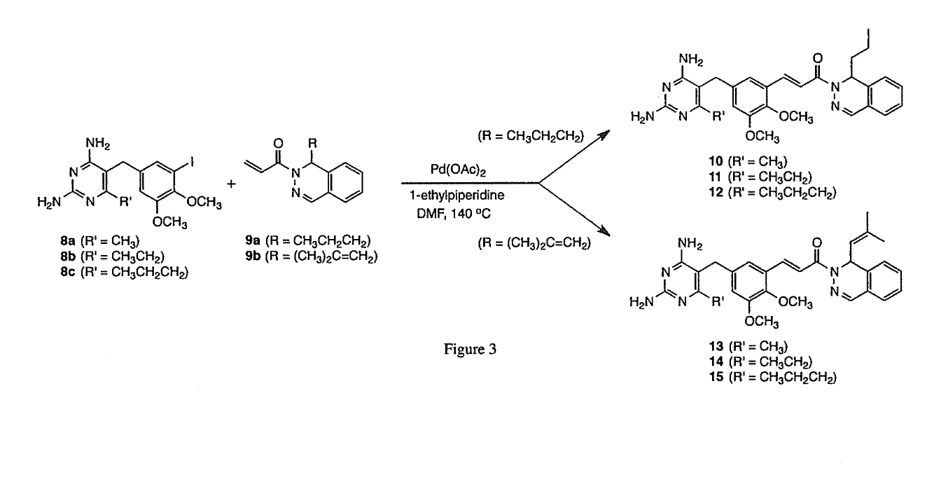

As noted above, FIG. 2 depicts an exemplary reaction scheme (Scheme 1) to produce intermediates 8a, 8b and 8c. The intermediates may then be converted to exemplary Formula 1 compounds 10-15 via, for example, reaction Scheme 2 shown in FIG. 3. Methods of manufacturing or producing the compounds using the schemes outlined in Schemes 1 and 2 are also provided by the invention. The methods include the general steps of combining, in a suitable solvent, i) a compound of Formula 2

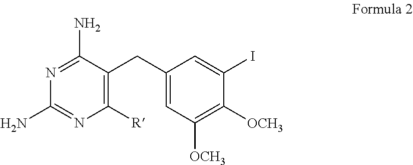

wherein R′ is selected from: C₁-C₆ alkyl or alkenyl groups with 1, 2, 3, 4, 5 or 6 carbon atoms, which may be: branched or unbranched; saturated or unsaturated; and may or may not be substituted; and ii) a compound of Formula 3,

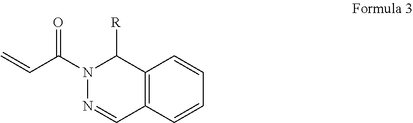

wherein R is selected from: C₁-C₆ alkyl or alkenyl groups with 1, 2, 3, 4, 5 or 6 carbon atoms, which may be: branched or unbranched; saturated or unsaturated; and may or may not be substituted. The step of combining is carried out under conditions that permit a reaction to occur between the compound of Formula 2 and the compound of Formula 3 to generate a compound of Formula 1. Exemplary suitable conditions for carrying out the reaction include but are not limited to: conducting the reaction in the presence of a catalyst (e.g. such as a Pd catalyst) and at a temperature of about 140° C. Suitable solvents include but are not limited to various organic and/or polar and/or aprotic solvents known in the art. Exemplary solvents include dimethylformamide, 1-ethylpiperidine of a combination of dimethylformamide and 1-ethylpiperidine.

The invention also provides novel compounds of Formula 2. A compound of Formula 2

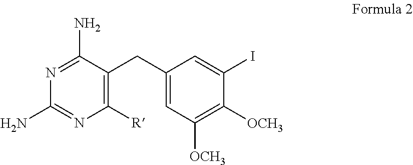

wherein R′ is selected from CH₃, CH₃CH₂ and CH₃CH₂CH₂.

### EXAMPLES

### Example I

### Synthesis of Exemplary Compounds

### Materials and Methods

Commercial anhydrous N,N-dimethylformamide (DMF) and dimethyl sulfoxide (DMSO) were stored under dry nitrogen and transferred by syringe into reactions where it was used. Tetrahydrofuran (THF) was dried over potassium hydroxide pellets and distilled from lithium aluminum hydroxide prior to use. Potassium carbonate (K₂CO₃) was heated at 120° C. under high vacuum for a period of 16 hours and stored in an over at 90° C. before use. All other commercial reagents were used as received.

Unless otherwise specified, all reactions were run under dry nitrogen in oven-dried glassware. Reactions were monitored by thin layer chromatography on silica gel GF plates (Analtech No. 21521). Preparative separations were performed by column chromatography on silica gel (grade 62, 60-200 mesh) mixed with UV-active phosphor (Sorbent Technologies, No. UV-5). Band elution was monitored using a hand-held UV lamp. The saturated NaCl, NH₄Cl and NaHCO₃ used in work-up procedure were aqueous solutions. Melting points were uncorrected. FT-IR spectra were run as thin films on sodium chloride disks. ¹H- and ¹³C-NMR spectra were measured on a Varian GEMINI 300 instrument at 300 MHz (¹H) and 75 MHz (¹³C), respectively, and referenced to internal tetramethysilance. Elemental analyses were performed by Atlantic Microlab, Inc., Norcross, Ga. 30071.

### 5-Iodo-3,4-dimethoxybenzaldehyde (2)

A procedure of Nimgirawath¹ was modified. A 250-mL, three-necked, round-bottomed flask, equipped with a magnetic stirrer, an addition funnel and a condenser was charged with 25.0 g (90 mmol) of 1, 100 mL of DMF and 37.0 g (0.27 mol) of anhydrous K₂CO₃ and stirred for 10 min. The reaction mixture was placed in a preheated oil bath at 120° C. for 15 min and 34.0 g (0.27 mol) of dimethyl sulfate added dropwise via a slow portion-wise process over 1 h. The reaction mixture was stirred at 120° C. for 18 h and then cooled. The mixture was cautiously added to 400 mL of distilled water and was stirred for 30 min. The crude product formed was collected and the product was recrystallized from 80:20 ethanol:water to give 25.2 g (96%) of 2 as a white solid, mp 71-72° C. (lit¹ mp 71-72° C.). IR: 2832, 2730, 2693 cm⁻¹; ¹H-NMR: δ 9.83 (s, 1H), 7.85 (d, 1H, J=1.7 Hz), 7.41 (d, 1H, J=1.7 Hz), 3.93 (s, 3H), 3.92 (s, 3H); ¹³C-NMR: δ 189.7, 154.2, 153.0, 134.7, 133.9, 111.0, 92.1, 60.7, 56.1.

### (3-Iodo-4,5-dimethoxyphenyl)methanol (3)

A method of Chowdhury and co-workers was modified.² A 250 mL, three-necked, round-bottomed flask, equipped with a magnetic stir bar and a condenser was charged with 2 (25 g, 85 mmol) and 100 mL of THF. Sodium borohydride (1.91 g, 50 mmol) was added slowly portion-wise over a period of 5 min and the reaction mixture was stirred at room temperature for 45 min. The reaction mixture was quenched with 100 mL of saturated NH₄Cl and extracted with ethyl acetate (3×125 mL). The combined organic layers were washed with saturated NaCl (100 mL), dried (MgSO₄) and concentrated under vacuum to yield 3 (24.8 g, 98.8%) as a thick colorless liquid. IR: 3392, 2824 cm⁻¹; ¹H-NMR (CDCl₃): δ 7.27 (d, J=1.6 Hz, 1H), 6.86 (d, J=1.6 Hz, 1H), 4.53 (s, 2H), 3.83 (s, 3H), 3.79 (s, 3H), 2.83 (br s, 1H); ¹³C-NMR (CDCl₃): δ 152.4, 147.7, 139.0, 128.3, 111.2, 92.1, 63.9, 60.3, 55.8.

### 5-(Bromomethyl)-1-iodo-2,3-dimethoxybenzene (4)

A 250-mL, three-necked, round-bottomed flask, fitted with a magnetic stirrer, an addition funnel and a condenser was charged with 3 (20 g, 0.068 mol) and 100 mL of dry ether. The vigorously stirred reaction mixture was cooled to 0° C. using an ice bath and phosphorus tribromide (20.2 g, 7.0 mL, 0.0748 mol, 1.1 equiv) was added dropwise to the reaction mixture over a period of 20 min. After addition, stirring was continued for an additional 30 min to ensure complete conversion. The reaction mixture was quenched by dropwise addition of 200 mL of saturated NaHCO₃ over a period of 45 min [Note: The quenching of the reaction mixture is done at a slower pace, faster addition leads to frothing of the organic layer from the reaction vessel.] The reaction mixture was transferred to a separatory funnel, the layers were separated and the aqueous layer was further extracted with ethyl acetate (3×150 mL). The combined extracts were further washed with saturated NaCl, dried (MgSO₄) and concentrated under vacuum to yield 4 (23.2 g, 96%) as a pale yellow solid, mp 64-65° C. IR: 2827 cm⁻¹; ¹H-NMR (CDCl₃): δ 7.37 (d, J=1.6 Hz, 1H), 6.91 (d, J=1.6 Hz, 1H), 4.40 (s, 2H), 3.87 (s, 3H), 3.83 (s, 3H); ¹³C-NMR (CDCl₃): δ 152.5, 149.0, 135.4, 130.7, 113.4, 92.2, 60.4, 56.0, 32.3.

### Ethyl 2-(3-Iodo-4,5-dimethoxybenzyl)-3-oxobutanoate (5a)

A method of Chowdhury and co-workers was modified.² A 250-mL, three-necked, round-bottomed flask, equipped with a magnetic stirrer, and a reflux condenser was charged with ethyl acetoacetate (8.75 g, 8.57 mL, 67.0 mmol) dissolved in 70 mL of dry ethanol. To the stirred solution, powdered sodium methoxide (3.63 g, 67.0 mmol) was added and the reaction mixture was warmed to 50° C. over a period of 30 min. To the warm mixture, compound 4 (20 g, 56.0 mol, 0.84 equiv) was added dropwise and the reaction was refluxed for 18 h. After cooling, the crude product was concentrated under vacuum and purified on a 100×3-cm silica gel column using 25% ethyl acetate in hexanes to give 6a (15.4 g, 68%) as colorless liquid. IR: 2828, 1736, 1716 cm⁻¹; ¹H-NMR (CDCl₃): δ 7.16 (d, J=1.6 Hz, 1H), 6.71 (d, J=1.6 Hz, 1H), 4.17 (q, J=7.3 Hz, 2H), 3.83 (s, 3H), 3.79 (s, 3H), 3.74 (t, J=7.3 Hz, 1H, 3.06 (m, 2H), 2.23 (s, 3H), 1.25 (t, J=7.3 Hz, 3H); ¹³C-NMR (CDCl₃): δ 201.9, 168.8, 152.3, 147.5, 136.2, 130.2, 113.5, 92.3, 61.6, 61.1, 60.3, 55.8, 32.9, 29.5, 14.0.

### Ethyl 2-(3-Iodo-4,5-dimethoxybenzyl)-3-oxopentanoate (5b)

The compound was prepared using the above procedure on a 56.0-mmol scale using ethyl 3-oxovalerate (9.69 g, 67.0 mmol) dissolved in 70 mL of dry ethanol, sodium methoxide (3.63 g, 67.0 mmol) and 4 (20 g, 56.0 mmol, 0.84 equiv) using the above procedure to obtain the product 6b (14.1 g, 60%) as a colorless liquid. IR: 2823, 1740, 1714 cm⁻¹; ¹H-NMR (CDCl₃): δ 7.15 (d, J=2.0 Hz, 1H), 6.69 (d, J=2.0 Hz, 1H), 4.16 (q, J=7.3 Hz, 2H), 3.82 (s, 3H), 3.79 (s, 3H), 3.74 (t, J=7.3 Hz, 1H), 3.06 (m, 2H), 2.62 (dq, J=18.1, 7.3 Hz, 1H), 2.39 (dq, J=18.1, 7.3 Hz), 1H), 1.23 (t, J=7.3 Hz, 3H), 1.03 (t, J=7.3 Hz, 3H); ¹³C-NMR (CDCl₃): δ 204.9, 168.9, 152.3, 147.6, 136.4, 130.3, 113.6, 92.3, 61.5, 60.3, 60.1, 55.9, 36.1, 33.2, 14.0, 7.5.

### Ethyl 2-(3-Iodo-4,5-dimethoxybenzyl)-3-oxohexanoate (5c)

The compound was prepared using the above procedure on a 56-mmol scale using ethyl butyrylacetate (10.6 g, 67.0 mmol) dissolved in 70 mL of dry ethanol, sodium methoxide (3.63 g, 67.0 mmol) and 4 (20 g, 56.0 mmol, 0.84 equiv) using the above procedure to obtain the product 4c (15.56 g, 64%) as a colorless liquid. IR: 2824, 1740, 1714 cm⁻¹; ¹H-NMR (CDCl₃): 7.15 (d, J=1.6 Hz, 1H), 6.70 (d, J=1.6 Hz, 1H), 4.16 (q, J=7.3 Hz, 2H), 3.82 (s, 3H), 3.79 (s, 3H), 3.74 (t, J=7.3 Hz, 1H), 3.06 (m, 2H), 2.55 (dt, J=17.6, 7.3 Hz, 1H), 2.36 (dt, J=17.6, 7.3 Hz, 1H), 1.56 (sextet, J=7.3 Hz, 2H), 1.23 (t, J=7.3 Hz, 3H), 0.86 (t, J=7.3 Hz, 3H); ¹³C-NMR (CDCl₃): δ 204.2, 168.7, 152.2, 147.5, 136.3, 130.2, 113.5, 92.2, 61.4, 60.3, 55.8, 44.5, 33.0, 16.7, 14.0, 13.4.

### 2-Amino-5-(3-iodo-4,5-dimethoxybenzyl)-6-methylpyrimidin-4-ol (6a)

To a solution of 5a (10.0 g, 24.6 mmol) in 30 mL of dry ethanol, guanidine carbonate (17.7 g, 98.5 mmol, 4 equiv) was added in a 100-mL single-necked, round-bottomed flask and the reaction mixture was allowed to reflux for a period of 18 h. The ethanol was concentrated to a minimal volume 50 mL of ice cold water was added and the reaction mixture was kept at 0° C. for 30 min to give a white precipitate. The solid was filtered and washed thoroughly with 100 mL of water, 50 mL of ether and then dried under high vacuum for 6 h to give 6a (7.40 g, 75%) as pure white solid, mp 185-186° C. IR: 3516-2358, 1654 cm⁻¹; ¹H-NMR (DMSO-d₆): δ 7.54 (br s, 1H), 7.08 (s, 1H), 6.97 (s, 1H), 6.58 (br s, 2H), 3.76 (s, 3H), 3.65 (s, 3H), 3.59 (s, 2H), 1.98 (s, 3H); ¹³C-NMR (DMSO-d₆): δ 169.6, 161.0, 158.4, 151.9, 145.9, 141.0, 128.6, 113.3, 108.6, 92.2, 59.7, 55.7, 29.8, 21.3.

### 2-Amino-5-(3-iodo-4,5-dimethoxybenzyl)-6-ethylpyrimidin-4-ol (6b)

The compound was prepared similarly on a 23.8-mmol scale from 5b (10.0 g) and guanidine carbonate (17.1 g, 95.2 mmol, 4 equiv) in 30 mL of dry ethanol to give 6b (7.70 g, 78%) as white solid, mp 190-191° C. IR: 3405-2390, 1666 cm⁻¹; ¹H-NMR (DMSO-d₆): δ 11.0 (br s, 1H), 7.07 (s, 1H), 6.94 (s, 1H), 6.54 (br s, 2H), 3.76 (s, 3H), 3.65 (s, 3H), 3.61 (s, 2H), 2.35 (q, J=7.1 Hz, 2H), 1.01 (t, J=7.1 Hz, 3H); ¹³C-NMR (DMSO-d₆): δ 167.2 (br), 163.8 (br), 153.9, 152.0, 146.2, 140.1, 128.6, 113.3, 109.0, 92.3, 59.8, 55.8, 28.8, 27.2 (br), 12.6.

### 2-Amino-5-(3-iodo-4,5-dimethoxybenzyl)-6-propylpyrimidin-4-ol (6c)

The compound was prepared similarly on a 23.0-mmol scale using 5c (10.0 g) and guanidine carbonate (16.6 g, 92.2 mmol, 4 equiv) in 30 mL of dry ethanol to give 6c (7.90 g, 80%) as white solid, mp 194-195° C. IR: 3520-2320, 1654 cm⁻¹; ¹H-NMR (DMSO-d₆): δ 7.16 (br s, 1H), 7.08 (s, 1H), 6.95 (s, 1H), 6.71 (br s, 2H), 3.76 (s, 3H), 3.65 (s, 3H), 3.61 (s, 2H), 2.29 (t, J=7.3 Hz, 2H), 1.45 (sextet, J=7.3 Hz, 2H), 0.84 (t, J=7.3 Hz, 3H); ¹³C-NMR (DMSO-d₆): δ 169.0, 164.2, 157.8, 151.9, 145.9, 141.3, 128.7, 113.3, 108.6, 92.2, 59.8, 55.7, 35.9, 29.4, 21.4, 14.0.

### 5-(3-Iodo-4,5-dimethoxybenzyl)-6-methylpyrimidine-2,4-diamine (8a)

A mixture of 6a (6.00 g, 15.0 mmol) in 15 mL of phosphorus oxychloride was refluxed (oil bath) over a period of 2 h. During this time, the suspension gradually becomes a brown homogenous solution. The reaction mixture was cooled in an ice bath for a period of 20 min and then slowly added into 150 g of ice dropwise with vigorous stirring to give a white precipitate. The solid was filtered under vacuum and washed thoroughly with 100 mL of water, 50 mL of a 20% ethanol water mixture and finally with 50 mL of ether to give 7a (5.7 g, 92%) as a white solid. This product was contaminated with several minor impurities and proved difficult to purify. Thus, it was carried on directly to the next step.

A stirred suspension of 7a (5.5 g, 13.0 mmol) in 80 ml dry ethanol was cooled to 0° C. and ammonia gas was bubbled through the solution for 15-20 min. The resulting solution was transferred into a pressure reactor and the heated to 165° C. for a period of 16 h. The reaction mixture was cooled and the solvent was evaporated under vacuum. The crude product was purified by flash chromatography on a 70×3-cm silica gel column eluted with dichloromethane:methanol:triethylamine (95:5:1) to give 8a (4.21 g, 81%) as white solid, mp 236-237° C. IR: 3420-2200, 1637 cm⁻¹; ¹H-NMR (DMSO-d₆): δ 6.99 (s, 1H), 6.91 (s, 1H), 6.51 (br s, 2H), 6.14 (br s, 2H), 3.77 (s, 3H), 3.69 (s, 2H), 3.65 (s, 3H), 2.08 (s, 3H); ¹³C-NMR (DMSO-d₆): δ 163.3, 159.6, 159.4, 152.0, 146.4, 138.3, 128.3, 113.4, 102.4, 92.6, 59.8, 55.8, 29.2, 20.1.

### 5-(3-Iodo-4,5-dimethoxybenzyl)-6-ethylpyrimidine-2,4-diamine (8b)

The compound was prepared on a 14.0-mmol scale from 6b (6.00 g) and 15 mL of phosphorus oxychloride to obtain 7b (5.61 g, 90%) as a brown solid. This product proved difficult to purify, and thus, was carried on directly to the next step.

A stirred suspension of 7b (5.50 g, 12.6 mmol) in 80 mL of dry ethanol was cooled to 0° C., treated as above with ammonia and heated to 165° C. for 16 h. Purification by flash chromatography gave 8b (4.20 g, 80%) as off white solid, mp 242-243° C. IR: 3460-2200, 1633 cm⁻¹; ¹H-NMR (DMSO-d₆): δ 6.98 (d, J=1.6 Hz, 1H), 6.91 (d, J=1.6 Hz, 1H), 6.57 (br s, 2H), 6.23 (br s, 2H), 3.77 (s, 3H), 3.72 (s, 2H), 3.65 (s, 3H), 2.41 (q, J=7.7 Hz, 2H), 1.04 (t, J=7.7 Hz, 3H); ¹³C-NMR (DMSO-d₆): δ 164.1, 163.6, 159.6, 152.0, 146.4, 138.5, 128.3, 113.4, 101.6, 92.5, 59.8, 55.8, 28.8, 25.8, 12.9.

### 5-(3-Iodo-4,5-dimethoxybenzyl)-6-propylpyrimidine-2,4-diamine (8c)

The compound was prepared on a 14.0-mmol scale from 6c (6.00 g) and 15 mL of phosphorus oxychloride to obtain 7c (5.70 g, 91%) as a brown solid. This product proved difficult to purify, and thus, was carried on directly to the next step.

A stirred suspension of 7c (5.50 g, 12.2 mmol) in 80 mL of dry ethanol was cooled to 0° C., treated as above with ammonia and heated to 165° C. for 16 h. Purification by flash chromatography gave 8c (4.31 g, 82%) as off white solid, mp 233-234° C. IR: 3480-2340, 1639 cm⁻¹; ¹H-NMR (DMSO-d₆): δ 6.98 (s, 1H), 6.91 (s, 1H), 6.39 (br s, 2H), 6.06 (br s, 2H), 3.77 (s, 3H), 3.72 (s, 2H), 3.66 (s, 3H), 2.36 (t, J=7.1 Hz, 2H), 1.49 (sextet, J=7.1 Hz, 2H), 0.84 (t, J=7.1 Hz, 3H); ¹³C-NMR (DMSO-d₆): δ 164.2, 163.4, 160.2, 151.9, 146.3, 138.8, 128.4, 113.4, 101.8, 92.5, 59.8, 55.8, 34.9, 29.0, 21.5, 13.9.

### (E)-3-(5-((2,4-Diamino-6-methylpyrimidin-5-yl)methyl)-2,3-dimethoxyphenyl)-1-(1-propyl-phthalazin-2(1H)-yl)prop-2-en-1-one (10)

To a stirred solution of 8a (1.00 g, 2.50 mmol) in dry DMF (8 mL), 1-(1-propylphthalazin-2(1H)-yl)prop-2-en-1-one³ (9a) (627 mg, 2.75 mmol, 1.1 equiv) dissolved in 1 mL of DMF was added, followed by the addition of 1-ethylpiperidine (310 mg, 0.38 mL, 2.75 mmol, 1.1 equiv) under nitrogen. To this, Pd(OAc)₂ (20 mg, 0.089 mmol) was added and the reaction mixture was heated at 140° C. for 20 h. The reaction mixture was purified by pouring the reaction mixture directly onto a 50×2.5-cm silica gel flash chromatography column and eluting with dichloromethane to remove impurities and finally with dichloromethane:methanol:triethylamine (97:3:1) to isolate the coupled product. This product was then subjected to a second flash chromatography eluted with dichloromethane:methanol:triethylamine (97:3:1) to remove colored impurities to give 10 (1.05 g, 84%) as a pale purple solid, mp 137-138° C. IR: 3329, 3185, 1651, 1616 cm⁻¹; ¹H-NMR (DMSO-d₆): δ 7.94 (s, 1H), 7.84 (d, J=15.9 Hz, 1H), 7.60 (d, J=15.9 Hz, 1H), 7.57-7.37 (complex m, 4H), 7.12 (s, 1H), 6.87 (s, 1H), 6.76 (br s, 2H), 6.28 (br s, 2H), 5.84 (t, J=6.6 Hz, 1H), 3.78 (s, 3H), 3.76 (s, 2H), 3.74 (s, 3H), 2.17 (s, 3H), 1.54 (m, 2H), 1.19 (m, 2H), 1.16 (t, J=7.1 Hz, 3H); ¹³C-NMR (DMSO-d₆): δ 165.5, 163.5, 158.5, 152.5, 146.0, 142.9, 136.7, 135.9, 133.6, 131.7, 128.3, 127.8, 126.5, 126.1, 123.6, 117.9 (2C), 114.0, 103.1, 60.7, 55.7, 50.3, 22.6, 19.7, 17.8, 15.2, 13.6 (one aromatic C unresolved). Anal. Calcd for C₂₈H₃₂N₆O₃.3.5H₂O.0.5 CH₃CH₂OH: C, 57.60; H, 6.33; N, 13.88. Found: C, 57.75; H, 6.43; N, 13.54.

### (E)-3-(5-((2,4-Diamino-6-ethylpyrimidin-5-yl)methyl)-2,3-dimethoxyphenyl)-1-(propyl-phthalazin-2(1H)-yl)prop-2-en-1-one (11)

The compound was prepared on a 2.42-mmol scale using 8b (1.00 g, 2.42 mmol), 9a (606 mg, 2.66 mmol, 1.1 equiv), Pd(OAc)₂ (20 mg, 0.089 mmol), and 1-ethylpiperidine (300 mg, 0.36 mL, 2.66 mmol, 1.1 equiv) dissolved in 9 mL of dry DMF under nitrogen atmosphere using the above procedure to obtain 11 (1.06 g, 85%) as a pale yellow solid, mp 192-193° C. IR: 3329, 3185, 1651, 1616 cm⁻¹; ¹H-NMR (DMSO-d₆): δ 7.94 (s, 1H), 7.84 (d, J=15.9 Hz, 1H), 7.61 (d, J=15.9 Hz, 1H), 7.57-7.36 (complex m, 4H), 7.12 (overlapping s, 2H and 1H), 6.88 (s, 1H), 6.60 (br s, 2H), 5.84 (t, J=6.6 Hz, 1H), 3.78 (overlapping s, 3H and 2H), 3.74 (s, 3H), 2.52 (obscured 2H), 1.53 (m, 2H), 1.18 (t, J=7.1 Hz, 3H), 1.16 (obscured, 2H), 1.10 (t, J=7.1 Hz, 3H); ¹³C-NMR (DMSO-d₆): δ 165.5, 164.1, 157.4, 152.5, 146.1, 142.9, 136.6, 135.6, 133.6, 131.7, 128.3, 127.9, 126.5, 126.1, 123.6, 118.0, 117.9, 114.0, 102.7, 60.8, 55.8, 50.3, 36.8, 29.0, 24.8, 17.8, 13.6, 12.8 (one aromatic C unresolved). Anal. Calcd for C₂₉H₃₄N₆O₃.5.0H₂O: C, 57.60; H, 6.33; N, 13.90. Found: C, 57.67; H, 6.33; N, 13.61.

### (E)-3-(5-((2,4-Diamino-6-propylpyrimidin-5-yl)methyl)-2,3-dimethoxyphenyl)-1-(1-propyl-phthalazin-2(1H)-yl)prop-2-en-1-one (12)

This compound was prepared on a 2.34-mmol scale using 8c (1.00 g, 2.34 mol), 9a (587 g, 2.57 mmol, 1.1 equiv), Pd(OAc)₂ (20 mg, 0.089 mmol), 1-ethylpiperidine (290 mg, 0.35 mL, 2.57 mmol, 1.1 equiv) dissolved in 9 mL of dry DMF under nitrogen atmosphere using the above procedure to obtain 12 (1.00 g, 81%) as an off white solid, mp 140-141° C. IR: 3335, 3190, 1650, 1615 cm⁻¹; ¹H-NMR (DMSO-d₆): δ 7.93 (s, 1H), 7.85 (d, J=15.9 Hz, 1H), 7.58 (d, J=15.9 Hz, 1H), 7.57-7.35 (complex m, 4H), 7.09 (s, 1H), 6.88 (s, 1H), 6.50 (br s, 2H), 6.09 (br s, 2H), 5.84 (t, J=6.6 Hz, 1H), 3.77 (overlapping s, 3H and 2H), 3.74 (s, 3H), 2.44 (t, J=7.1 Hz, 2H), 1.53 (m, 4H), 1.16 (m, 4H), 0.87 (t, J=7.1 Hz, 3H), 0.82 (t, J=7.1 Hz, 3H); ¹³C-NMR (DMSO-d₆): δ 165.5, 163.6 (2C), 159.6, 152.4, 145.9, 142.8, 136.6, 136.4, 133.6, 131.7, 128.2, 127.8, 127.8, 126.5, 126.1, 123.6, 117.9, 117.7, 114.1, 102.4, 60.8, 55.7, 50.3, 36.8, 34.7, 29.5, 21.5, 17.8, 13.9, 13.6. Anal. Calcd for C₃₀H₃₆N₆O₃.2.75H₂O: C, 62.32; H, 6.92; N, 14.32. Found: C, 62.26; H, 6.69; N, 14.06.

### (E)-3-(5-((2,4-Diamino-6-methylpyrimidin-5-yl)methyl)-2,3-dimethoxyphenyl)-1-(isobutenylphthalazin-2(1H)-yl)prop-2-en-1-one (13)

The compound was prepared on a 2.50 mmol scale using 8a (1.00 g, 2.50 mmol), (±)-1-(1-isobutenyl-2(1H)-pthalazinyl)-2-propen-1-one³ (9b) (660 mg, 2.75 mmol, 1.1 equiv), Pd(OAc)₂ (20 mg, 0.089 mmol), and 1-ethylpiperidine (310 mg, 0.38 mL, 2.75 mmol, 1.1 equiv) dissolved in 9 mL of dry DMF under nitrogen atmosphere using the above procedure to obtain 13 (1.02 g, 80%) as an as a pale off-white solid, mp 165-166° C. IR: 3333, 3186, 1652, 1618 cm⁻¹; ¹H-NMR (DMSO-d₆): δ 7.93 (s, 1H), 7.83 (d, J=15.9 Hz, 1H), 7.56 (d, J=15.9 Hz, 1H), 7.52 (m, 2H), 7.43 (d, J=7.1 Hz, 1H), 7.30 (d, J=7.1 Hz, 1H), 7.11 (s, 1H), 6.96 (br s, 2H), 6.86 (s, 1H), 6.52 (br s, 2H), 6.49 (d, J=9.9 Hz, 1H), 5.24 (d, J=9.9 Hz, 1H), 3.78 (s, 3H), 3.76 (s, 2H), 3.73 (s, 3H), 2.19 (s, 3H), 1.96 (s, 3H), 1.60 (s, 3H); ¹³C-NMR (DMSO-d₆): δ 165.2, 163.7, 157.8, 152.5, 146.1, 142.1, 136.8, 135.6, 133.8, 133.5, 132.2, 128.2, 127.9, 126.3, 126.2, 123.1, 122.1, 118.0, 117.9, 114.0, 103.3, 60.7, 55.7, 49.2, 29.5, 25.3, 19.1, 18.4 (one aromatic C unresolved). Anal. Calcd for C₂₈H₃₂N₆O₃.2.5H₂O.0.5 CH₃CH₂OH: C, 62.15; H. 6.94; N, 14.47. Found: C, 62.44; H, 7.04; N, 14.60.

### (E)-3-(5-((2,4-Diamino-6-ethylpyrimidin-5-yl)methyl)-2,3-dimethoxyphenyl)-1-(isobutenylphthalazin-2(1H)-yl)prop-2-en-1-one (14)

The compound was prepared on a 2.42-mmol scale using 8b (1.00 g, 2.42 mmol), 9b (639 mg, 2.66 mmol, 1.1 equiv), Pd(OAc)₂ (20 mg, 0.89 mmol), and 1-ethylpiperidine (300 mg, 0.36 mL, 2.66 mmol, 1.1 equiv) dissolved in 9 mL of dry DMF under nitrogen atmosphere using the above procedure to obtain 14 (1.06 g, 84%) as a pale yellow solid, mp 192-193° C. IR: 3329, 3182, 1651, 1615 cm⁻¹; ¹H-NMR (DMSO-d₆): δ 7.91 (s, 1H), 7.84 (d, J=15.9 Hz, 1H), 7.53 (d, J=15.9 Hz, 1H), 7.50 (m, 2H), 7.43 (d, J=7.1 Hz, 1H), 7.30 (d, J=7.1 Hz, 1H), 7.07 (s, 1H), 6.88 (s, 1H), 6.49 (d, J=9.9 Hz, 1H), 6.29 (br s, 2H), 5.91 (br s, 2H), 5.24 (d, J=9.9 Hz, 1H), 3.77 (s, 3H), 3.76 (s, 2H), 3.73 (s, 3H), 2.45 (q, J=7.3 Hz, 2H), 1.96 (s, 3H), 1.60 (s, 3H), 1.07 (t, J=7.3 Hz, 3H); ¹³C-NMR (DMSO-d₆): δ 166.0, 165.2, 163.4, 160.6, 152.4, 145.9, 142.1, 136.8, 136.7, 133.8, 133.5, 132.2, 128.2, 127.8, 126.3, 126.2, 123.1, 122.2, 117.8, 117.6, 114.1, 101.6, 60.8, 55.7, 49.2, 29.5, 26.4, 25.3, 18.4, 13.0. Anal. Calcd for C₃₀H₃₄N₆O₃.2.0H₂O: C, 64.04; H, 6.81; N, 14.90. Found: C, 64.05; H, 6.72; N, 14.64.

### (E)-3-(5-((2,4-Diamino-6-propylpyrimidin-5-yl)methyl)-2,3-dimethoxyphenyl)-1-(isobutenylphthalazin-2(1H)-yl)prop-2-en-1-one (15)

The compound was prepared on a 2.34-mmol scale using 8c (1.00 g, 2.34 mmol), 9b (617 mg, 2.57 mmol, 1.1 equiv), Pd(OAc)₂ (20 mg, 0.089 mmol), and 1-ethylpiperidine (290 mg, 0.35 mL, 2.57 mmol, 1.1 equiv) dissolved in 9 mL of dry DMF under nitrogen atmosphere using the above procedure to obtain 15 (980 mg, 78%) as a pale white solid, mp 138-139° C. IR: 3340, 3177, 1656, 1606 cm⁻¹; ¹H-NMR (DMSO-d₆): δ 7.90 (s, 1H), 7.84 (d, J=15.9 Hz, 1H), 7.50 (m, 3H), 7.43 (d, J=7.1 Hz, 1H, 7.30 (d, J=7.1 Hz, 1H), 7.05 (s, 1H), 6.88 (s, 1H), 6.48 (d, J=9.9 Hz, 1H), 6.03 (br s, 2H), 5.69 (br s, 2H), 5.24 (d, J=9.9 Hz, 1H), 3.76 (s, 3H), 3.73 (overlapping s, 2H and 3H), 2.39 (t, J=7.1 Hz, 2H), 1.95 (s, 3H), 1.59 (s, 3H), 1.52 (sextet, J=7.1 Hz, 2H), 0.86 (t, J=7.1 Hz, 3H); ¹³C-NMR (DMSO-d₆): δ 166.4, 165.2, 163.2, 161.4, 152.4, 145.9, 142.1, 137.0, 136.8, 133.8, 133.5, 132.2, 128.2, 127.8, 126.24, 126.17, 123.1, 122.2, 117.8, 117.6, 11.42, 101.8, 60.8, 55.7, 49.2, 35.7, 29.8, 25.3, 21.6, 18.4, 14.1. Anal. Calcd for C₃₁H₃₆N₆O₃.0.75H₂O: C, 66.41; H, 7.18; N, 14.99. Found: C, 66.26; H, 6.91; N, 14.74.

### Example 2

### Determination of the Minimum Inhibitory Concentration (MIC)

Experiments with were carried out with B. anthracis Sterne and Staphylococcus aureus 29213 in a BSL-2 laboratory with appropriate biosafety and security measures. The MICs were determined using a broth microdilution assay in accordance with the Clinical and Laboratory Standards Institute (CLSI) recommendations (CLSI. 2009. Methods for dilution antimicrobial susceptibility tests for bacteria that grow aerobically; approved standard, 8th ed., vol. 29. Clinical Laboratory Standards Institute, Wayne, Pa.).

Each MIC was determined in duplicate for two replicates; 96-well plates containing 2-fold serial dilutions of test compounds or commercial antibiotics (used for quality control as directed by CLSI guidelines CLSI. 2010. Performance standards for antimicrobial susceptibility testing, 20th information update, vol. 29. Clinical Laboratory Standards Institute, Wayne, Pa.) were prepared in cation-adjusted Mueller-Hinton broth for all agents. Ten microliters of an inoculum standardized to 0.5 McFarland units was used to infect wells containing 100 microliters of medium with or without drug.

Inoculum concentrations were verified by plating for colony forming units (CFU) using the CLSI recommended protocol (CLSI. 2009. Methods for dilution antimicrobial susceptibility tests for bacteria that grow aerobically; approved standard, 8th ed., vol. 29. Clinical Laboratory Standards Institute, Wayne, Pa.). Experimental controls included growth control wells, sterility control wells, and uninoculated drug wells to verify that test compounds remained soluble under experimental conditions. Experiments were incubated at 37° C. for 16 h (B. anthracis) or 18 h (S. aureus). After the appropriate incubation, plates were allowed to equilibrate to room temperature for 30 minute they were then sealed, and the absorbance at 600 nm was spectrophotometrically measured to determine the MIC, defined as the lowest compound concentration that inhibited growth of the microorganism. Visual confirmation of growth was also performed.

When variation was obtained in experimental values the MIC is reported as a range. Growth patterns with some S. aureus strains exhibited trailing, as has been previously noted for folate pathway inhibitors. In these instances, MIC values were calculated as the concentration of compound resulting in an 80% reduction in growth of the microorganism (CLSI. 2009. Methods for dilution antimicrobial susceptibility tests for bacteria that grow aerobically; approved standard, 8th ed., vol. 29. Clinical Laboratory Standards Institute, Wayne, Pa.). However, this did not result in a shift of a MIC from that obtained using the lowest concentration of compound that inhibited growth of the microorganism. See Table A.

### Enzyme Preparation and Determination of the IC₅₀.

The gene for the dihydrofolate reductase (DHFR) protein was cloned from genomic material, purified from B. anthracis Sterne or S. aureus and placed in a pET101D vector (Invitrogen). This allows recombinant protein production in E. coli BL21 (DE3) cells and encodes an additional six histidine residues and linker at the C-termini of the protein. Purification utilized immobilized metal ion affinity chromatography (GE Life Sciences) using nickel charged resin to chelate with the added histidine residues. Purification of eluted protein culminated with an S100 size exclusion column (GE Life Sciences) with a running buffer of 20 mM Tris (pH 8), 150 mM NaCl, and 5% glycerol.

Purified recombinant DHFR proteins were stored at −80° C. in 10% glycerol; the presence of the histidine tag did not affect activity. The enzymatic assay was adapted from the standard format (Barrow, E. W., P. C. Bourne, and W. W. Barrow. 2004). Functional cloning of Bacillus anthracis DHFR and confirmation of natural resistance to trimethoprim. Antimicrobial Agents Chemother. 48:4643-4649.) to a high-throughput 96-well plate platform with a 200 microliter total reaction volume and was carried out with a Biomek 2000 liquid handling robot interfaced with a DTX880 plate reader. Enzyme, saturating co-factor (NADPH), and inhibitor in dimethyl sulfoxide were preincubated at 30° C.; the reaction was initiated by the addition of dihydrofolic acid (DHF) and monitored for 3 min, during which time the reaction remained linear. The protein concentration is adjusted to yield a specific activity of 1.4 nmol DHF reduced per minute per mg of DHFR. Detection utilized the redox-sensitive tetrazolium dye 3-(4,5-dimethylthiazol-2-yl)-5-(3-carboxymethoxyphenyl)-2-(4-sulfophenyl)-2H-tetrazolium (MTS). MTS is reduced by the product tetrahydrofolate to yield an increased absorbance at 450 nm. Reactions were performed in at least triplicate. The change in signal was calculated as a percentage as compared to a reaction with no inhibitor over 2.8 min of reaction, and these values were used to calculate an absolute IC₅₀ from the fit of a four-parameter logistic model using the KC Jr. plate reader software.

Related to these methods are those disclosed by Bourne, C. R., E. W. Barrow, R. A. Bunce, P. C. Bourne, K. D. Berlin, and W. W. Barrow, Inhibition of antibiotic-resistant Staphylococcus aureus by the broad-spectrum dihydrofolate reductase inhibitor RAB1. Antimicrobial Agents and Chemotherapy, 2010. 54(9): 3825-3833, the contents of which are herein incorporated by reference.

The results of the MIC determinations are presented in Table 1. As can be seen, all of the compounds listed in Table 1 inhibited the anthrax bacillus and the target enzyme, DHFR, suggesting that they should be useful in the treatment of anthrax infections following exposure to the anthrax spores.

TABLE 1
Inhibitory Properties of the Highly Substituted
2,4-Diaminopyridimidines

| Compound | MIC (mg/mL) | IC₅₀ (nM) |
|---|---|---|
| 10 | 8 | 516.5 |
| 11 | 4 | 17390 |
| 12 | 1 | 17140 |
| 13 | 4 | 101.8 |
| 14 | 4 | 181.1 |
| 15 | 2 | 18000 |

(MIC=Minimum Inhibitory Concentration in milligrams per milliliter)
(IC₅₀=Inhibitory Concentration at nanomolar concentration where 50% of growth inhibition occurred with the bacteria colonies.)

### REFERENCES

1. Nimgirawath, S. Aust. J. Chem. 1994, 47, 725-731.
2. Chowdhury, S. F.; Guerrero, R. H.; Brun, R.; Ruiz-Perez, L. M.; Pacanowska, D. G.; Gilbert, I. H. J. Enzyme Inhibition and Med. Chem. 2002, 17, 293-302.
3. Nammalwar, B.; Bunce, R. A.; Berlin, K. D.; Bourne, C. R.; Bourne, P. C.; Barrow, E. W.; Barrow, W. W. Unpublished results.

Thus, the present invention is well adapted to carry out the objects and attain the ends and advantages mentioned above as well as those inherent therein. While presently preferred embodiments have been described for purposes of this disclosure, numerous changes and modifications will be apparent to those of ordinary skill in the art.

What is claimed is:

1. A compound of Formula 1: 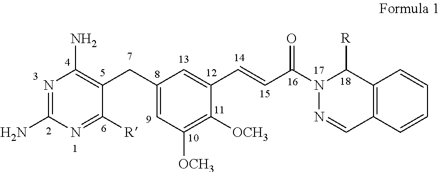
wherein
R and R′ may be the same or different and are independently selected from: C₁-C₆ alkyl or alkenyl groups with 1, 2, 3, 4, 5 or 6 carbon atoms, which may be: branched or unbranched; saturated or unsaturated; and may or may not be substituted;
and isomers, pharmacologically acceptable salts, solvates, and hydrates thereof.

2. The compound of claim 1, wherein said R and R′ are selected from methyl, ethyl, propyl, isopropyl, n-butyl, isobutyl, sec-butyl, tert-butyl, n-pentyl, isopentyl, neopentyl, hexyl, 2-methylpentyl, 3-methylpentyl, 2,3-dimethylbutyl; 2,2-dimethylbutyl, vinyl groups and allyl groups.

3. The compounds of claim 1, wherein R is n-propyl or isobutenyl.

4. The compound of claim 1, wherein R′ is methyl, ethyl or n-propyl.

5. The compound of Formula 1, wherein the compound is selected from the group consisting of:

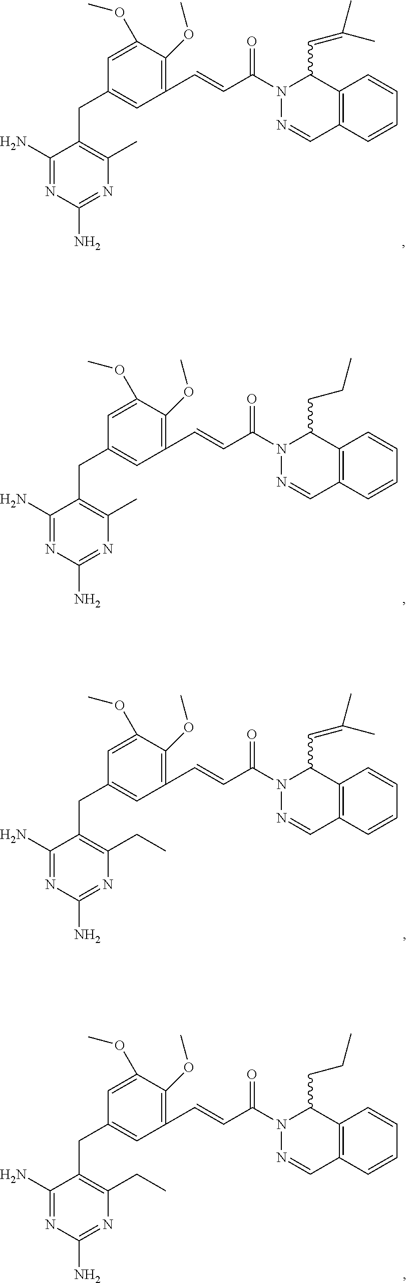 [SMILES: COc1cc(Cc2c(C)nc(N)nc2N)cc(/C=C/C(=O)N2N=Cc3ccccc3C2C=C(C)C)c1OC ; CCCC1c2ccccc2C=NN1C(=O)/C=C/c1cc(Cc2c(C)nc(N)nc2N)cc(OC)c1OC ; CCc1nc(N)nc(N)c1Cc1cc(/C=C/C(=O)N2N=Cc3ccccc3C2C=C(C)C)c(OC)c(OC)c1 ; CCCC1c2ccccc2C=NN1C(=O)/C=C/c1cc(Cc2c(N)nc(N)nc2CC)cc(OC)c1OC]

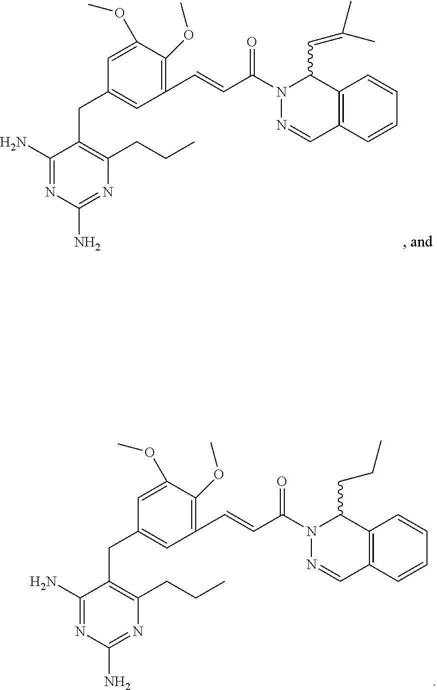 [SMILES: CCCc1nc(N)nc(N)c1Cc1cc(/C=C/C(=O)N2N=Cc3ccccc3C2C=C(C)C)c(OC)c(OC)c1 ; CCCc1nc(N)nc(N)c1Cc1cc(/C=C/C(=O)N2N=Cc3ccccc3C2CCC)c(OC)c(OC)c1]

6. A method of preventing or treating an anthrax infection in a subject in need thereof, comprising
administering to the subject a therapeutically effective amount of a compound of Formula 1: 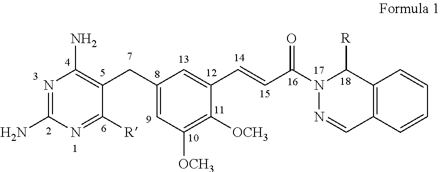
wherein
R and R′ may be the same or different and are independently selected from: C₁-C₆ alkyl or alkenyl groups with 1, 2, 3, 4, 5 or 6 carbon atoms, which may be: branched or unbranched; saturated or unsaturated; and may or may not be substituted; or an isomer, pharmacologically acceptable salt, solvate, or hydrate thereof; and
a pharmaceutically compatible carrier.

7. The method of claim 6, wherein said R and R′ are selected from methyl, ethyl, propyl, isopropyl, n-butyl, isobutyl, sec-butyl, tert-butyl, n-pentyl, isopentyl, neopentyl, hexyl, 2-methylpentyl, 3-methylpentyl, 2,3-dimethylbutyl; 2,2-dimethylbutyl, vinyl groups and allyl groups.

8. The method of claim 6, wherein R is n-propyl or isobutenyl.

9. The method of claim 6, wherein R′ is methyl, ethyl or n-propyl.

10. The method of claim 6, wherein the compound is selected from the group consisting of:

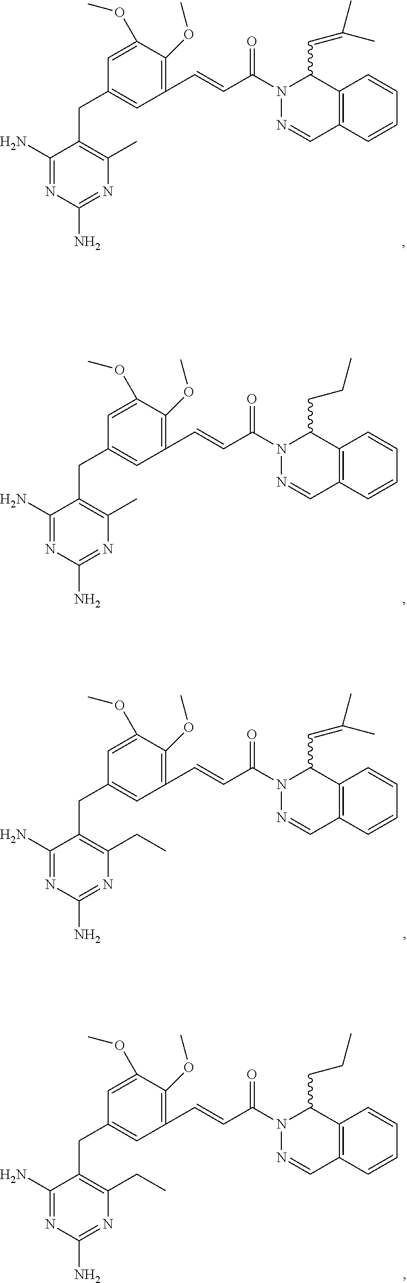 [SMILES: COc1cc(Cc2c(C)nc(N)nc2N)cc(/C=C/C(=O)N2N=Cc3ccccc3C2C=C(C)C)c1OC ; CCCC1c2ccccc2C=NN1C(=O)/C=C/c1cc(Cc2c(C)nc(N)nc2N)cc(OC)c1OC ; CCc1nc(N)nc(N)c1Cc1cc(/C=C/C(=O)N2N=Cc3ccccc3C2C=C(C)C)c(OC)c(OC)c1 ; CCCC1c2ccccc2C=NN1C(=O)/C=C/c1cc(Cc2c(N)nc(N)nc2CC)cc(OC)c1OC]

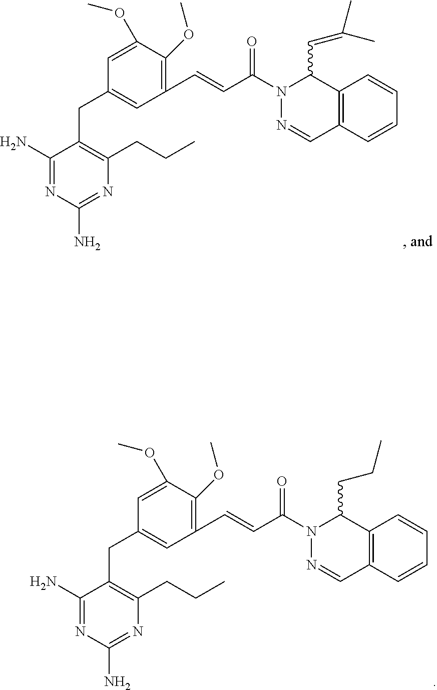 [SMILES: CCCc1nc(N)nc(N)c1Cc1cc(/C=C/C(=O)N2N=Cc3ccccc3C2C=C(C)C)c(OC)c(OC)c1 ; CCCc1nc(N)nc(N)c1Cc1cc(/C=C/C(=O)N2N=Cc3ccccc3C2CCC)c(OC)c(OC)c1]

11. A method of killing Bacillus anthracis, comprising
contacting said Bacillus anthracis with a lethal amount of a compound of Formula 1: 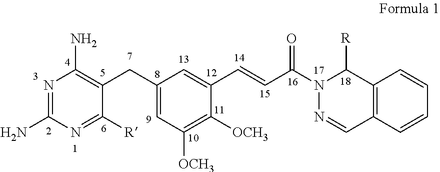
wherein
R and R′ may be the same or different and are independently selected from: C₁-C₆ alkyl or alkenyl groups with 1, 2, 3, 4, 5 or 6 carbon atoms, which may be: branched or unbranched; saturated or unsaturated; and may or may not be substituted; or an isomer, pharmacologically acceptable salt, solvate, or hydrate thereof.

12. A method of inhibiting dihydrofolate reductase (DHFR), comprising
contacting said DHFR with an amount of a compound of Formula 1: 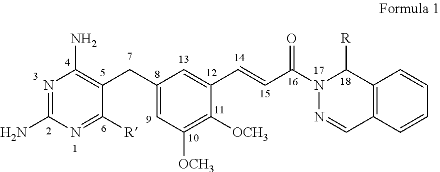
wherein
R and R′ may be the same or different and are independently selected from: C₁-C₆ alkyl or alkenyl groups with 1, 2, 3, 4, 5 or 6 carbon atoms, which may be: branched or unbranched; saturated or unsaturated; and may or may not be substituted; or an isomer, pharmacologically acceptable salt, solvate, or hydrate thereof;
wherein said amount is sufficient to inhibit said DHFR.

13. A method of synthesizing a compound of Formula 1, 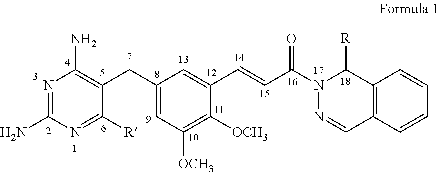
wherein
R and R′ may be the same or different and are independently selected from: C₁-C₆ alkyl or alkenyl groups with 1, 2, 3, 4, 5 or 6 carbon atoms, which may be: branched or unbranched; saturated or unsaturated; and may or may not be substituted; or an isomer, pharmacologically acceptable salt, solvate, or hydrate thereof; said method comprising
combining, in a suitable solvent,
i) a compound of Formula 2 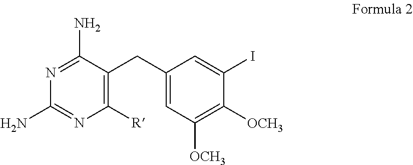
wherein
R′ is selected from: C₁-C₆ alkyl or alkenyl groups with 1, 2, 3, 4, 5 or 6 carbon atoms, which may be: branched or unbranched; saturated or unsaturated; and may or may not be substituted; and
ii) a compound of Formula 3, 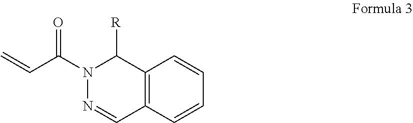
wherein
R is selected from: C₁-C₆ alkyl or alkenyl groups with 1, 2, 3, 4, 5 or 6 carbon atoms, which may be: branched or unbranched; saturated or unsaturated; and may or may not be substituted;
wherein said step of combining is carried out under conditions that permit a reaction to occur between said compound of Formula 2 and said compound of Formula 3 to generate said compound of Formula 1.

14. The method of claim 13, wherein said conditions include carrying out said reaction in the presence of a catalyst and at a temperature of 140° C.

15. The method of claim 14, wherein said catalyst is a Pd catalyst.

16. The method of claim 13, wherein said suitable solvent is dimethylformamide, 1-ethylpiperidine of a combination of dimethylformamide and 1-ethylpiperidine.

17. The method of claim 13, wherein R and R′ are selected from methyl, ethyl, propyl, isopropyl, n-butyl, isobutyl, sec-butyl, tert-butyl, n-pentyl, isopentyl, neopentyl, hexyl, 2-methylpentyl, 3-methylpentyl, 2,3-dimethylbutyl; 2,2-dimethylbutyl, vinyl groups and allyl groups.

18. The method of claim 13, wherein R is n-propyl or isobutenyl.

19. The method of claim 13, wherein R′ is methyl, ethyl or n-propyl.

20. A compound of Formula 2 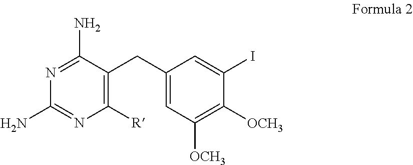
wherein R′ is selected from CH₃, CH₃CH₂ and CH₃CH₂CH₂.
